(19) United States
(12) Patent Application Publication
(10) Pub. No.: US 2008/0089810 A1
(43) Pub. Date: Apr. 17, 2008

(54) Universal Regulatory Apparatus for Controlled Environments for Use with Added Sensors and Analyzers

(76) Inventor: John R. B. Lighton, Las Vegas, NV (US)

Correspondence Address: THE LUTHER LAW FIRM, 12198 E. COLUMBINE DR., SCOTTSDALE, AZ, 85259, US

(21) Appl. No.: 11/549,974

(22) Filed: Oct. 17, 2006

(51) Int. Cl. G01N 33/00

(52) U.S. Cl. 422/83

(57) ABSTRACT

A gas-based regulatory apparatus has an electrical input, optionally connected to an analog to digital converter; a central processing unit (CPU) comprising a non-volatile memory for storing and retrieving program and configuration data and a random access memory for storing and retrieving other data; a visible alphanumeric display means connected thereto; a user input means adjacent to the display and connected to the processing means; an alarm driven directly or indirectly from the CPU; a pneumatic valve having an input port and an output port, and being driven directly or indirectly from the CPU; and a program that runs on the CPU, for receiving user input, measuring input voltages and pressure voltages from the analog to digital converter, displaying data, converting measured voltages to gas and pressure units, implementing a control algorithm, and driving the pneumatic valve and alarm.

### TECHNICAL FIELD

This invention relates to a universal regulatory apparatus which utilizes a microcomputer to control a parameter in a controlled environment with the parameter data provided by a separate user-added sensor or analyzer. More particularly, the invention accepts sensor and analyzer input in a voltage rage of <1 mV to 5V, transforms raw voltage into numbers with relevant units, and increases flexibility of operation, thus making it readily adaptable to frequent changes in gas regulation experiments.

### BACKGROUND

Many biological laboratories study the effects of changes in gas composition on organisms in enclosures. The laboratories have ready sources of different gases (oxygen, carbon dioxide, helium, carbon monoxide, nitrogen, etc.) and gas sensors or analyzers. A number of devices for controlling oxygen or carbon dioxide concentration are available on the commercial market. They function by adding a controlling gas to a sealed or semi-sealed environment, the interior of which is monitored for the gas in question, or for a different gas that is diluted by the controlling gas (such as O₂ diluted by N₂). The available systems vary considerably in capability and complexity.

For example, the Biospherix company (www.biospherix.com) lists a number of basic or advanced controllers specifically meant to regulate O₂ or CO₂, or both. Basic models have very limited input/output capabilities. More advanced models have any of a variety of different gas analyzers built-in at time of order for controlling different gases, and some are capable of advanced operations such as computer control or even generating custom ramps. U.S. Patent Publication 20030092178 describes an aquatic rather than an aerial gas analyzer and is intended for tissue-culture work.

In another example, Sable Systems International (www.sablesys.com) produces an eight-channel controller with an integral paramagnetic auto-calibrating oxygen analyzer, intended for O₂ regulation only. Several other companies, such as Coy Labs, produce dedicated O₂ and/or CO₂ controllers with built-in analyzers. All of the controllers made by the above manufacturers utilize Proportional-Integral-Derivative (PID) control for optimally smooth, fast-responding control of the target gas concentration; all are either fixed O₂ and/or CO₂ analyzers or need to be ordered specifically for use with another specified gas or gases.

Such existing art fulfills the need for gas regulation in fixed systems where the controlled gases, and their likely concentration ranges, are known at time of purchase. It is however necessary in many busy research laboratories to regulate a particular gas for specific projects for brief periods—hours to months. However, on the next day or week, the specific gas to be controlled is likely to change from, for example, O₂ to CO₂ to CO to NO to H₂O vapor. In such laboratories dedicated analyzers are already available; but controllers that can be quickly reconfigured to operate with different analyzers, and to display their different readings in suitable units such as percent concentration or ppm, and which additionally are optimized to control gas parameters, do not exist. Likewise, commercial voltage-producing sensors yield a voltage proportional to the concentration of a target gas, such as fuel cell sensors for O₂. Critically, such sensors often produce very low voltages—such as 10 mV at 21% O₂—that cannot be read reliably by a controller designed for the far larger (typically 1-5V) outputs of other gas analyzers, even if a general purpose controller could be found.

What is needed is a versatile gas-based regulator suitable for the ever-shifting requirements of a busy research laboratory and having the following characteristics at a minimum. First, it would be capable of accurate control with primary sensors and gas analyzers having an output range from the low millivolts up to at least 5V, with high accuracy and resolution throughout the whole range. Second, it would have a display and user input controls that allow it to be self-sufficient, with no need for a computer umbilical in basic operational mode. Third, it would transform voltage inputs to user-specified units meaningful to the user, such as % O₂ or kPa H₂O. Fourth, it would utilize PID control in order to achieve smooth control in spite of the significant lag times often characteristic of gas analyzers, especially those remote from the controlled environment. Preferably, such a device would also allow a) logging or recording of the controlled data and b) remote setting of parameters such as setpoint for externally generated ramps or profiles. The regulator would also have alarm capabilities, so that users can be alerted to common problems, such as a low control-gas tank pressure.

Extensive investigation in physiological research has yielded no versatile (i.e., not dedicated to a specific gas) regulators available with the above characteristics. Such an apparatus would be appreciated as unique and invaluable in many research laboratories.

### SUMMARY OF THE INVENTION

In one embodiment, there is provided a gas-based regulatory apparatus, that comprises an electrical input, optionally connected to an analog-to-digital converter; a central processing unit (CPU) comprising a non-volatile memory for storing and retrieving program and configuration data and a random access memory for storing and retrieving other data; a visible alphanumeric display means connected thereto; a user input means adjacent to the display and connected to the processing means; an alarm driven directly or indirectly from the CPU; a pneumatic valve having an input port and an output port, and being driven directly or indirectly from the CPU; and a program that runs on the CPU, for receiving user input, measuring input voltages and pressure voltages from the analog to digital converter, displaying data, converting measured voltages to gas concentration and pressure units, implementing a control algorithm, and driving the pneumatic valve and alarm.

In another embodiment, the gas-based regulatory apparatus has the pneumatic valve input port connectable to a user-supplied controlling gas source. A user-supplied gas source can be connected via a pressure regulator to the pneumatic valve input port. The pneumatic valve input port can be connected to nitrogen to regulate the oxygen content of a vessel in the hypoxic region. The pneumatic valve input port can be connected to oxygen to regulate the oxygen content of a vessel in the hyperoxic region. The pneumatic valve input port can be connected to carbon dioxide to regulate the carbon dioxide content of a control vessel in the hypercapnic region. Any other gas can likewise be controlled in this fashion. In addition, the apparatus has an internal pressure means for monitoring the constant availability of the gas. The input port of the pneumatic valve is connected to the pressure measurement means.

In another embodiment, the gas-based regulatory apparatus has user input means that are connected to the CPU such that numerical parameters, operating mode, and other relevant data can be entered into the memory of the CPU.

In yet another embodiment, during normal operation, the visual display of the gas-based regulatory apparatus shows the control setpoint and the present value of the controlled parameter. The visual display additionally shows input gas line pressure and valve drive. The visual display shows a menu, when appropriate, to instruct the operator in the entry of at least one parameter. The menu instructs the operator in the entry of a setpoint using the user input means.

In yet another embodiment, the gas-based regulatory apparatus receives input from a sensor or analyzer that measures a variable that the user desires to control, with the analyzer or sensor either being within or sampling from within a sealed or semi-sealed vessel within which the variable is to be controlled. The electrical input from the sensor or analyzer can be internally connected to a programmable gain amplifier (PGA), the gain of which is controlled via the CPU, and wherein an algorithm running in the program maximizes the resolution of the measured input voltage. The algorithm can maximize the measured voltage to a full scale range. The algorithm can accept data between about 5 mV and about 5 V full scale, thereby enabling the use of raw oxygen fuel cell or other sensors or conventional analyzers. The measured value from the sensor or analyzer is optionally acquired in digital form via a digital data link. The measured input voltage can be interactively transformed, via the user input means and the program, so that displayed units are familiar to the user. The input voltage can vary from 0-5V and is converted to and displays 0-100% O₂. The transformed input voltage can be trimmed at the zero point and at a span point by interactive use of the user input means, program and display means. The control algorithm running in the program can read the transformed output of the attached analyzer or sensor, compare it to a user-entered setpoint, and implement a control algorithm of the Proportional Integral Derivative (PID) type.

In another embodiment, the voltage input may be replaced by or augmented with a digital input capable of receiving information sent from a sensor or analyzer in digital form such as, by way of non-limiting example, an RS-232 serial link.

In yet another embodiment, the gas-based regulatory apparatus has a program that controls the amount of the control gas per unit time passing through the pneumatic valve into a control vessel in accordance with the degree of delivery of the controlling gas dictated by the control algorithm.

In another embodiment, the gas-based regulatory apparatus has the operator input accepting an alarm threshold for the variable being controlled, such that when the variable strays from the upper or lower alarm threshold, the alarm is activated. A plurality of alarm thresholds can be entered. The digital output means for retrieving data can transfer the data to an external memory. The apparatus can have a digital input means or serial port for connecting the apparatus to an external computer, thereby enabling remote entry of data such as control setpoint and alarm thresholds.

In another embodiment, the gas-based regulatory apparatus has a driver for the pneumatic valve, the driver being connected to the CPU, thereby enabling a routine in the memory to detect an open circuit fault programmatically and optionally activate the alarm.

In another embodiment, the gas-based regulatory apparatus provides for the zero and span of the pressure reading to be interactively adjusted by the user, using the user input and display means, such that the display reads 0 at 0 applied pressure to the control gas input port on the pneumatic valve, and the display reads the correct pressure when a known pressure is applied, with the sensitivity of the pressure measurement means being, in a preferred embodiment, approximately 0-300 kPa.

### BRIEF DESCRIPTIONS OF THE DRAWINGS

The above noted and other features of the invention will be better understood from the following detailed description when considered with reference to the accompanying drawings.

FIG. 1 is an overview diagram of the apparatus.

FIG. 2 is a schematic diagram of the detail of the programmable gain amplifier (PGA) shown in FIG. 1 and other adjustments that can be made to the input to be regulated and displayed.

FIG. 3 is a diagram illustrating the set up of the apparatus for regulation of oxygen in a controlled environment, using external fuel cell sensor, gas supply and optional fan.

FIG. 4 is a diagram illustrating the set up of the apparatus for regulation of oxygen, carbon dioxide or other gas in a controlled environment, using external user-supplied analyzer and gas supply.

FIG. 5 is a diagram illustrating the use of the apparatus for regulation of dissolved oxygen in a controlled aquatic environment with external user-supplied aquatic gas analyzer and gas supply.

FIG. 6 is a diagram illustrating the use of the apparatus for pressure regulation in a controlled environment with external user-supplied pressure meter and vacuum or pressure source.

### DETAILED DESCRIPTION

This universal regulatory apparatus provides a versatile, microcomputer-based gas controller. It is designed primarily to regulate oxygen and carbon dioxide concentrations. Nevertheless, the apparatus can also control practically any variable for which (a) a sensor or analyzer exists, and (b) a gas stream or pressure source will effect a change in the variable. The Figures and Examples illustrate only some of the possible uses.

The apparatus can be used with almost any kind of sensor. Any instrument, analyzer or sensor with a voltage data output between 1 mV and 5V can be used to supply electrical input that the inventive apparatus receives, reports and uses to control the desired parameter. The apparatus is auto-ranging, automatically adjusting its internal voltage measurement circuitry to optimize its resolution, which in a preferred embodiment is a high 24 bits. The apparatus can be used directly with almost any commercially available fuel cell oxygen sensor. Such sensors generally produce only about 8-15 mV in air, or about 40-75 mV at 100% oxygen.

The user interface is uncluttered but allows maximum flexibility. All operational parameters are accessible from an alphanumeric display on the front of the apparatus. Adjustments are made with drift-less digital controls and maintained in non-volatile memory that has a rated retention of decades. Because the operating system is built in, no dedicated external software packages are required to input user-specified parameters or to calibrate the instrument. The user interface is designed for quick and easy calibration and control of gas concentration within a closed loop control system. If the fuel cell is properly calibrated after attachment to the apparatus, using the fuel cell with this regulatory apparatus provides greater accuracy than most gas-mixing systems.

A minimum of moving parts adds to the robustness of the apparatus. There are built-in diagnostic routines that continuously monitor the apparatus in operation. The diagnostics begin with a thorough systems check at turn-on and are programmed to run at frequent intervals after that, thus, ensuring the integrity of scientific experiments and the safety of any research organism(s). An alarm sounds if a problem is detected. In addition, upper and lower limit alarms for the regulated parameter can be specified.

The apparatus also is pre-programmed for advanced Proportional-Integral-Derivative (PID) control. This type of control provides minimal overshoot and undershoot for the most stable regulation of the selected parameter. Because of the limitation on over- and under-shoot, very little gas is used after the setpoint is reached, unless a disturbance occurs, whereupon a controlled amount of gas is pulsed into the system only until the setpoint is reached.

The output for record-keeping or recording is the regulated parameter data as an analog voltage readable by any data acquisition program. The data are also available as a serial data stream. In addition, the display shows both the setpoint and current reading.

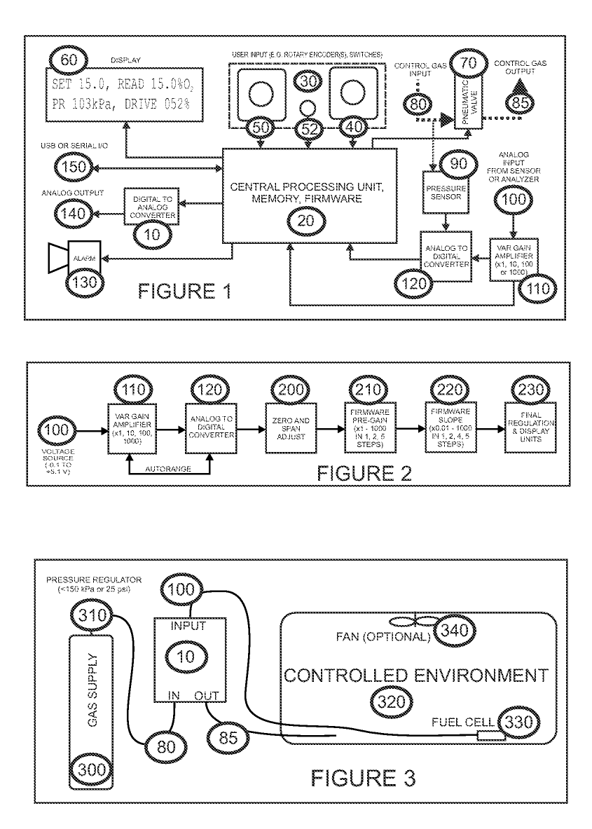

Referring now to FIG. 1, the versatile gas-based regulator (“apparatus”) 10 is shown in conceptual diagram form. Electrical connections are shown as solid lines with an arrowhead denoting the direction of information transfer. Plumbing is denoted by dotted lines, and grouping of components with a conceptual block is denoted by dashed lines. In one embodiment, the apparatus requires a 11.5 to 14V DC source at 20 mA to 200 mA operating current, depending on whether the gas solenoid valve is on or off and whether display backlighting is selected.

The central processing unit (CPU) 20 contains, or has access to, memory both volatile and non-volatile. The latter stores firmware that provides the instrument's operating intelligence and the durable storage of user-entered operating parameters. The volatile memory provides the fast “scratch-pad” memory required by the firmware. Such CPUs are often, but are not necessarily, integrated onto one chip complete with all required memory, a good example being the PIC18F4550 (Microchip).

The CPU 20 communicates directly or indirectly with all of the other elements of FIG. 1. Via the user interface 30, the user can input operating parameters such as, by way of non-limiting example, the control setpoint; the units of the setpoint and the controlled variable such as % O₂ or % CO₂; and the transformations required to transform a given voltage input to yield meaningful figures. User input is performed interactively in user input block 30, in one embodiment via a menu system implemented with a rotary encoder 40 and a push-button switch 50. User-entered parameters are stored in non-volatile memory.

The display 60 provides a menu and visual feedback during entry of operating parameters, and also serves to communicate the instrument's operational status (setpoint, current value of the controlled variable, etc.) during normal operation. In a preferred embodiment display 60 is an alphanumeric LCD or PLED 2-line by 16-character display, but other display modalities, such as graphics arrays, are equally feasible.

A pneumatic or gas solenoid valve 70 is operated by a driver (not shown) driven from the CPU 20, in a preferred embodiment via a switching transistor or FET. The valve 70 is driven by Pulse Width Modulation (PWM) so as to progress from a completely shut-off configuration (modulation=0%) to a completely open configuration (modulation=100%) or anywhere in between according to the output of the CPU 20. The function of the pneumatic valve 70, in a preferred embodiment, is to allow a modulated flow of a gas from a gas input 80 into a controlled environment vessel (not shown) within which the controlled variable is being regulated (see FIG. 3, below). The control gas affects the controlled variable positively (if it is the variable the concentration of which is being controlled, such as O₂ or CO₂) or negatively (if it dilutes a controlled gas, such as N₂ diluting O₂). In an alternative embodiment, the control gas input 80 may be replaced with a positive or negative pressure source relative to ambient pressure, in order to effect pressure control within a vessel. In one embodiment, the valve 70 is limited to a pressure less than 150 kPa.

The pneumatic valve 70 is the effector in a classic control loop circuit. The CPU 20 is the integrator of information in that control loop; its firmware compares a user-entered setpoint against the measured control variable, and drives the valve 70 according to the deviation of the measured control variable from the setpoint. In a preferred embodiment, the CPU 20 also takes into account the time for which the control variable has deviated from the setpoint and the current rate of change of the control variable. These three parameters are assigned multipliers (gain, reset and derivative) to suit a given control situation. In a preferred embodiment, the firmware implements the integration of sensor information, setpoint, and valve PWM drive by using the well known Proportional Integral Derivative (PID) control algorithm, which accounts for the three integration parameters mentioned above. In a preferred embodiment the three PID parameters (gain, reset and derivative) are adjustable by the user, using the display 60 and the user input section 30 in conjunction with the CPU 20. All user-entered PID values are stored in the non-volatile memory of the CPU 20.

Depending on the direction of regulation (increase or decrease of the controlled variable with addition of the control gas), the user, in a preferred embodiment, can set the direction of regulation, which is equivalent to the sign of the gain term mentioned in the previous paragraph. For example, addition of nitrogen reduces the concentration of oxygen in a chamber; the user specifies downward regulation (using the display 60 and the user input section 30 in conjunction with the CPU 20) which in effect gives the gain a negative sign. The user-entered regulation direction is stored in the non-volatile memory of the central processing unit 01. In the case of regulation of O₂ (as determined by the units being % O₂) each setpoint is, in a preferred embodiment, examined and if it is below ambient O₂ levels (i.e. <20.9% O₂), the regulation direction is automatically assumed to be negative and vice versa. This assumption may be overridden by the user.

We now require the operator to connect a sensor or analyzer (not shown) to inform the control loop's integrator of the value of the controlled variable. The external analog input 100 accepts analog signals from most voltage-generating sensors, meters and analyzers. Such a device serves as the sensor in the control loop and is supplied by the user of the instrument. In preferred embodiments, the sensor can be an oxygen fuel cell sensor (typical output 10 mV at 21% O₂); or alternatively, an analyzer having an output of e.g. 0-5 V for 0-100% O₂ or a CO₂ analyzer having a voltage output of e.g. 0-5 V for 0-5% CO₂ might be used. Thus, it is evident that the external analog input can vary over a wide range, ca. 1000-fold.

Accordingly, the external analog input voltage is amplified by the programmable gain amplifier (PGA) 110, with the CPU 20 able to vary the gain of the PGA, in a preferred embodiment, over a range of ×1-×1000. Suitable PGA chips include the Texas Instruments PGA204, the gain of which can be varied via a two-bit control line over a range of 1-1000× in tenfold steps.

The CPU 20 reads the amplified voltage using the analog to digital converter (ADC) 120, which in a preferred embodiment is a 24-bit overranging sigma-delta ADC such as the Linear Technologies LTC2400. Such ADCs have an input range from about −0.2V to +5.2V; alternatively any conventional ADC may be used. The voltage is read preferably on a regular basis, with a preferred inter-reading interval of about 200 milliseconds. If the ADC reads an over-range voltage (the ADC is at its maximum reading), and if the gain of the PGA is set to greater than 1, the firmware reduces the PGA gain until the signal comes into range. Alternatively, if the ADC reads an under-range voltage (less than approximately 10% of its full-scale input voltage), and if the gain is set to less than 1000, the firmware increases the PGA gain until the signal achieves acceptable amplitude. This process maximizes the resolution of the ADC and facilitates optimal readings of the controlled variable from practically any sensor or analyzer.

In an alternative embodiment, the instrument may obtain data from the sensor or analyzer via a digital data connection implemented, by way of non-limiting example, with a serial data link using the RS-232 protocol. In a given embodiment such a digital data link may augment or even replace the above-described analog input and associated circuitry.

In many preferred embodiments of the instrument it is important to sense the input pressure of the control gas being passed through pneumatic valve 70. This is because excessively low pressure, resulting from a mis-adjusted gas pressure regulator or an empty tank, may cause control to fail. Thus a pressure sensor 90 is employed to sense the pressure at the input gas line of the pneumatic valve 70. In a preferred embodiment the pressure sensor 90 is a gauge sensor having a range of approximately 0-300 kPa, or in general a range exceeding the maximum operating pressure of the pneumatic valve 70.

The pressure sensor's output is amplified (if required) by, in a preferred embodiment, an instrumentation amplifier such as the Texas Instruments INA122. The resulting voltage is multiplexed into the ADC 120 so that it can be read by the CPU 20. Within the firmware the resulting ADC reading is converted to a pressure reading in the scratchpad memory of the CPU 20, using a user-determined conversion factor. In a preferred embodiment, the zero and span of the pressure readings are adjustable by the user, using the display 60 and the user input section 30 in conjunction with the CPU 20. Zero and span adjustment coefficients are stored in the non-volatile memory of the CPU 20.

In another embodiment the pressure sensor may possess a digital output directly readable by the CPU 20 without requiring external amplification or an ADC. An example of a suitable sensor of this type is the All Electronics 30 PSI-G-DO.

As mentioned above, the apparatus 10 has an alarm 130, which the CPU 20 triggers when certain parameters are exceeded; these parameters can be adjusted by the operator. In this embodiment, the apparatus 10 is further equipped with two outputs, an analog output 140 and a USB or serial I/O port 150. Internally connected to the analog output 140 can be a digital-to-analog converter 160, or this function can be incorporated into the firmware.

A number of conditions merit alerting the user of the instrument that a critical problem may exist. These may include, but are not necessarily limited to, excessively low or high control gas pressure, and/or the controlled variable drifting beyond an acceptable upper or lower limit. For that reason, in a preferred embodiment an alarm 130 can sound to alert the user. In a preferred embodiment, the alarm 130 may be acoustic (e.g., Mallory Sonalert) and may be supplemented in a given embodiment with a visual alarm and/or a relay. Typically such an alarm or alarms 130 will be driven indirectly from the CPU 20 via a switching transistor or FET.

Should the input pressure of the control gas fall below a critical value, in a preferred embodiment the alarm 130 can be activated. The pressure threshold for sounding the alarm, in a preferred embodiment, is adjustable by the user, using the display 60 and the user input section 30 in conjunction with the CPU 20. In a preferred embodiment, an upper pressure limit may also be entered, and the alarm capability may also be shut off completely, as not all applications will require it. All user-entered alarm pressure thresholds are stored in the non-volatile memory of the central processing unit 01.

The alarm 130 may also, in a preferred embodiment, serve as notice that the controlled variable has left an acceptable window between an upper and a lower alarm threshold, such thresholds in a preferred embodiment being adjustable by the user, using the display 60 and the user input section 30 in conjunction with the CPU 20. In a preferred embodiment, the alarm capability may also be shut off completely, as not all applications will require it. All user-entered controlled-variable alarm thresholds are stored in the non-volatile memory of the CPU 20.

In addition, the alarm 130 may also, in a preferred embodiment, serve as notice that the pneumatic valve 70 is open-circuit or disconnected, or that the transistor or FET driving the pneumatic valve 70 has shorted or developed an open circuit fault. By monitoring the voltage across the valve driver transistor or FET, using a digital input to the CPU 20 in series with a current limiting resistor, the firmware can test whether the expected voltage is present under full-on or full-off PWM drive conditions. For example, if the valve 70 is turned on but the voltage across the driving transistor or FET remains high, the driving transistor or FET has developed an open circuit fault. If the valve 70 is turned off but the voltage across the driving transistor or FET remains low, the driving transistor or FET has developed a short circuit fault or the valve has developed an open circuit fault, and so on. In a preferred embodiment this alarm 130 can be turned on or off, using the display 60 and the user input section 30 in conjunction with the CPU 20. The active or inactive status of this alarm 130 is stored in the non-volatile memory of the CPU 20.

All instruments that control variables should provide some form of output in order to allow users to log the values of the controlled variable over time. Accordingly, the apparatus 10 in a preferred embodiment allows the CPU 20 to send a digital value to a digital-to-analog converter 160, connected to an analog output connector 140. In a preferred embodiment, the user, using the display 60 and the user input section 30 in conjunction with the CPU 20, can set the analog output 140 to yield a voltage corresponding to a defined part of the range of the controlled variable. For example, the user may specify that the range of 0-100% or 0-50% of the controlled variable's possible range of values be represented by an analog output of 0-5V. In addition, the serial output 150 can in a preferred embodiment be configured to produce loggable output strings at a user-settable interval, for receipt and storage using a terminal program. All user-entered output parameters are stored in the non-volatile memory of the CPU 20.

Although all relevant operating parameters (setpoint, alarm thresholds, PID coefficients, etc.) are adjustable by the user, using the display 60 and the user input section 30 in conjunction with the CPU 20, in a preferred embodiment at least a subset of these operating parameters are also changeable via remote input, in a preferred embodiment via a serial or USB connection 150 that allows direct communication with the CPU 20. For example, the PIC18F4550 CPU contains support for both serial and USB communications. In such an embodiment the instrument may be directed by the user, using the display 60 and the user input section 30 in conjunction with the CPU 20, to enter a command-driven mode. The operating mode is stored in the non-volatile memory of the CPU 20. In command mode, in a preferred embodiment, current values of the controlled variable and the setpoint may be obtained on command, and operating parameters may be entered remotely by sending a one-letter mnemonic such as “S” for setpoint, followed by the numeric value to be applied to that parameter. Because most non-volatile memory technologies allow only a limited number of writes (typically 100,000) before errors occur, and because remotely entered parameters may change very frequently, in a preferred embodiment the instrument does not store remotely entered parameters in non-volatile memory, although it does so store whether or not it is configured to operate in command mode.

We now return to considerations of the conversion of measured input voltages into the units that will be displayed and regulated. Within the firmware of the CPU 20, the numeric output of the ADC 120 is converted to an internal representation of the voltage input to the ADC 120. The internal representation is stored in scratchpad memory, in a preferred embodiment as a floating point number having at least 4 bytes of precision. The resulting voltage reading is then divided by the gain setting of the PGA 110 to yield the voltage at the analog input 100 and is also stored in similar format in the scratchpad memory. This constitutes the unprocessed voltage input that must then be converted to user units, and which must also be adjustable in terms of both zero and slope(=span) so that the user can fine-tune the instrument's reading to agree with a given external analyzer's reading or a given sensor's calibration.

Turning now to FIG. 2, this illustrates the steps in converting voltage input 100 into the final regulation and display units on the display 60 and in the output, such as analog output 140. The apparatus 10 receives analog input from the sensor 100 in the form of voltage, which travels to the PGA 110. Between the PGA 110 and the ADC 120, an autorange is established in firmware for the input 100 from the sensor. The next processing step 200 consists of adjustment of the zero and span to allow fine-tuning of the input data so that analyzer or sensor data are read correctly. Next, the firmware retrieves the pre-gain 210 and the slope 220 of the data, which are also processed by the firmware (see below for more details). The final process is the regulation 230 of the valve 70, so that the proper amount of gas, pressure, etc. is dispensed to maintain the feedback loop in the desired state. The regulation 230 activates a drive (not shown) that operates the valve 70. Regulation step 230 also actuates the display 60 to show current values for the programmed parameter.

In the preferred embodiment, the apparatus 10 affords the operator opportunities to change settings. The operator interface 30 has three controls arranged in a straight line below the display 60 in the order (from left to right) MODE 50, ENTER 52 and ADJUST 40. The MODE and ADJUST knobs (50 and 60, respectively) are connected to internal rotary encoders that click when the knobs turn for audible and tactile feedback. The ENTER switch 52 is connected to an internal pushbutton that also provides audible and tactile feedback.

MODE 50 provides access to all the user-changeable parameters, including setpoint adjustment and external instrument or fuel cell sensor selection. When the operator clicks through the available MODEs, each is displayed on the 2-line LCD display 60. Values are changed by turning the ADJUST knob 40, and the changed value is displayed. Clockwise movement increases the value and vice versa. Faster turning of the ADJUST knob 40 makes the values change more rapidly. At the maximum for a value, it automatically scrolls to its minimum, and vice versa. Registration and storing of values is accomplished by pressing the ENTER button 52; the value is stored in non-volatile memory. For a change of value without storing, the MODE knob 50 is turned one click in any direction without pressing ENTER. This cancels the adjustment. The apparatus 10 is returned to its factory-default settings by turning it off and then holding down ENTER while turning it on. RESETTING appears in the display to confirm activation of the operator's command.

In use, first, the mode is set: fuel cell or analyzer. With fuel cell selected, the units are automatically oxygen percent. For all other analyzers, the units are selectable by the user at the user input 30.

In a preferred embodiment the instrument's zero and span settings 200 are adjustable by the user, using the display 60 and the user input section 30 (via the menu options) in conjunction with the CPU 20. The zero adjustment in a preferred embodiment adds a value which may be positive or negative to the measured voltage such that when the attached sensor or analyzer is reading zero, the instrument's reading is zero as well. The span adjustment multiples the measured voltage by a factor that in a preferred embodiment is adjustable over the range of approximately 0.5-2.0, such that at a given known input value (such as 20.9% O₂ to a fuel cell oxygen sensor in air) the instrument's readings can be adjusted to yield an identical value. All user-entered zero and span parameters are stored in the non-volatile memory of the CPU 20.

Next, the zero and span corrected input voltage is, in a preferred embodiment, multiplied by a user-enterable constant in step 210, over a typical range of 1-1000 in 1-2-5 steps, referred to as pre-gain for reasons explained below. This maximizes flexibility of embodiment by allowing an instrument's or sensor's output to be multiplied so that it falls into the nominal range of 0-5V. For example, an oxygen fuel cell may produce 10 mV at 20% O₂. Multiplying this figure by a pre-gain of 100 means that the fuel cell (after appropriate zero and span correction in step 2003) yields an effective output, for purposes of further calculation, of 1.00 V at 20% O₂ or 0-5V for 0-100% O₂.

“Pre-gain” refers simply to the fact that the gain is applied (in firmware) prior to the calculation of the final user value by application of a firmware slope in step 2205. That slope is a factor, in a preferred embodiment variable over the range 0.01 to 1000 by user input in 1-2-4-5 steps that converts the analog input of the instrument after zero and span adjustment and pre-gain application into final user inputs. In the example in the previous paragraph a gain of 20 would be applied. Finally, an appropriate set of units in step 230 is selected, in a preferred embodiment from a selection of common units such as % O₂, % CO₂, % CO, % CH₄, kPa, ppm, ° C. and so on. All user-entered pre-gain, slope and unit selections are stored in the non-volatile memory of the CPU 20.

The following examples serve to clarify the above-described steps. (1) For a CO₂ analyzer with an analog output of 0-5V for 0-5% CO₂, set the units to CO₂%, the pre-gain to 1, and the slope to 1 (5/5). The resulting regulation and display units are % CO₂. (2) For an O₂ analyzer with an analog output of 0-5V for 0-100% O₂, set the units to O₂%, the pre-gain to 1, and the slope to 20 (100/5; other combinations of pre-gain and slope that are factors of 20 will also work). The resulting regulation and display units are % O₂. (3) For an O₂ analyzer with an analog output of 0-5V for 0-25% O₂, set the pre-gain to 1, and set the slope to 5 (25/5). The resulting regulation and display units are % O₂. (4) For a fuel cell with a nominal output of 50 mV for 100% O₂, set the pre-gain to 100, and the slope to 20 (100/(0.05*100)). Sensor-to-sensor variations can be accounted for with the firmware zero and span controls 200. Note that this last example is also, in a preferred embodiment, available as a preset option for use with fuel cells only.

In an alternative embodiment, the user would enter, using the display 60 and the user input section 30 in conjunction with the CPU 20, a value equivalent to that displayed on the analyzer or being measured by the sensor. The firmware would then assign appropriate pre-gain and slope values automatically and store them in the non-volatile memory of the CPU 20.

In cases where no exact combination of pre-gain and slope exists to transform an instrument's or sensor's voltage output to the appropriate units, the zero and span adjustments 200 serve to fine-tune the transformation of voltage inputs into user units. Thus, almost all commonly used transformations between input voltages and user units are accommodated.

In another embodiment, a digital data (rather than analog data) connection to a sensor or analyzer may yield data in voltage or in final user units such as % O₂. The above-described transformation scheme is flexible enough to allow either case to yield the desired data in the instrument.

In a preferred embodiment the setpoint is user-adjustable, using the display 60 and the user input section 30 in conjunction with the CPU 20, or by using the remote inputs 150, over the range of 0-100% of the range of the attached instrument or sensor, with a resolution of 0.1% or better. In a preferred embodiment, the setpoint is expressed directly in user units, not as percentages of instrument range, except of course in the case where instrument units are percentages. All user-entered setpoints are stored in the non-volatile memory of the CPU 20.

In operation, as disclosed above, the setpoint and the present reading, both in familiar user units, are compared using the PID algorithm, and the pneumatic valve 70 is driven accordingly. In a preferred embodiment the pneumatic valve 70 is of the on-off style and is driven via slow PWM, over a typical period of approximately 1-2 seconds per cycle, and is turned on for a percentage of time per cycle dictated by the PWM algorithm. By way of example, if the PID algorithm dictates 50% PWM drive, and the cycle duration is 1 second, the pneumatic valve is turned on for 0.5 second and off for 0.5 second, once per second. Likewise, if the PID algorithm dictates 91% PWM drive, and the cycle duration is 1 second, the pneumatic valve is turned on for 0.91 second and off for 0.09 second, once per second.

In an alternative embodiment, the same PWM modulation as described above may be used but instead of driving a pneumatic valve, a conventional or solid state relay can be utilized to control an effector such as a heating coil.

In an alternative embodiment, higher frequency PWM (typically ca. 19 kHz) is used to control a pneumatic valve of the proportional style such that it is completely closed at 0% PWM and completely open at 100% PWM.

The foregoing descriptions of the preferred embodiments of the present invention have been presented for purposes of illustration and description. They are not intended to be exhaustive or to limit the invention to the precise forms disclosed, and obviously many other modifications and variations are possible in light of the above teachings. The embodiments were chosen and described to best explain the principles of the invention and its practical applications, thereby enabling others skilled in the art to best utilize the invention in its various embodiments and with various modifications as are suited to the particular use contemplated. It is intended that the scope of the invention be defined by the following claims, including all equivalents.

### EXAMPLES

### Example 1

### Using the Apparatus to Regulate Oxygen Using a Fuel Cell Sensor

FIG. 3 is a schematic of the set up of this experiment. The gas supply (oxygen, nitrogen, helium, etc.) 300 provides gas to the apparatus 10 at a pressure of no more than 150 kPa, usually through a pressure regulator 310. The pressure regulator 310 has an outlet which is connected to gas inlet 80. For regulation of oxygen above 21% oxygen, the gas supply 300 is oxygen. For regulation of oxygen below 21%, the gas supply 400 is any diluent gas such as nitrogen or helium. Generally, the smaller the enclosure 320, the lower the required pressure for efficient regulation. The fuel cell 330 should be positioned in an open area of the controlled enclosure, away from sudden temperature shifts. An optional fan 340 to stir the air will assure that the air is well mixed, assisting in the speed and stability of regulation. The fuel cell 330 is calibrated before being connected to the inventive apparatus 10 at input 100.

### Example 2

### Using the Apparatus to Regulate Oxygen or Other Gas Using an Analyzer

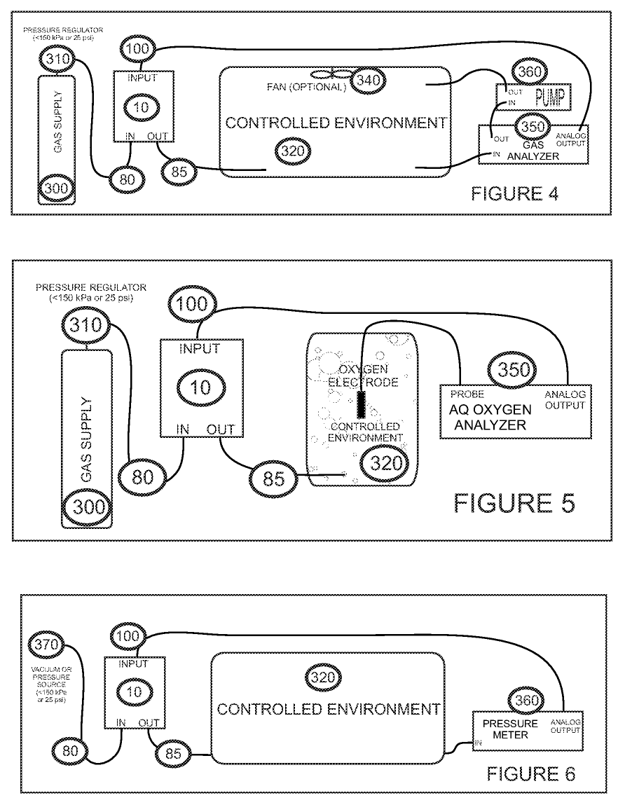

FIG. 4 shows the general set up for controlling oxygen, carbon dioxide, carbon monoxide, etc. using a specific gas analyzer. This is very similar to the fuel cell oxygen regulation set up, except that (a) an external analyzer is used, and (b) the gas being controlled in not necessarily oxygen. The gas could be carbon dioxide, water vapor (using a bubbler on the output line from the apparatus), carbon monoxide, methane, etc. FIG. 4 additionally shows a pump 360 (which may be internal or external to the analyzer, depending on the analyzer make and model). The pump 360 is required to give adequate sample flow for most analyzers except, e.g., hygrometers with an external probe. One example of a suitable sampling pump is the Sable Systems SS-3 subsampler. The tubing to the external analyzer should be kept as short as practical, with a rather high flow rate in the sampling circuit (typically about 200-1000 ml/min). If the analyzer takes a long time to respond to changes in gas concentrations, regulation by the inventive apparatus may suffer, producing over-or under-shoot. This can be mitigated by reducing the PID gain. In this set up, the settings of the inventive apparatus may need to be changed. For example, the operator needs to input the analyzer type, change the regulation setpoint, determine convenient units and possibly change the PID gain.

### Example 3

### Using the Apparatus to Regulate Dissolved Oxygen Using an Analyzer

FIG. 5 shows the set up for controlling dissolved oxygen in a controlled aquatic environment 320 using a user-supplied aquatic gas analyzer 350. In this modality air (for increasing dissolved oxygen) or nitrogen (for reducing dissolved oxygen) is added to the water by bubbling, probably via an air stone. This alters the measured oxygen concentration in the correct direction and allows control via the inventive apparatus. Note that building gas supply 300 is compressed air that probably needs a regulator 310 to drop its pressure below 150 kPa. In many instances, a needle valve (not shown) also is needed to reduce flow to the air stone in the controlled environment 320. Such a needle valve should be connected before the inventive apparatus 10. Various settings of the apparatus 10 need to be configured for the inventive apparatus to regulate this set up, starting with the input type of analyzer.

### Example 4

### Using the Apparatus to Regulate Pressure Using a Pressure Meter

FIG. 6 shows the set up for controlling pressure in a controlled environment 320 using a pressure meter 360. In this modality, the apparatus 10 is connected to a vacuum or pressure source 370 to provide higher or lower pressure than ambient, depending on whether the situation is intended to be hypobaric or hyperbaric. Building-supplied pressure or vacuum are best suited, provided that positive pressure does not exceed 150 kPa. If the pressure changes too rapidly, a needle valve should be used to reduce flow into or out of the enclosure 320. Such a needle valve should be connected between the pressure source 370 and the inventive apparatus 10. Starting the settings with the input of analyzer, the operator then configures the controls for this set up.

Although a preferred embodiment of the invention has been described with some particularity, many modifications and variations in the invention are possible within the light of the above teachings. Therefore, it is to be understood that, within the scope of the appended claims, the invention may be practiced other than as specifically described.

1. A gas-based regulatory apparatus, comprising:
a. an electrical input, optionally connected to an analog to digital converter;
b. a central processing unit (CPU) comprising a non-volatile memory for storing and retrieving program and configuration data and a random access memory for storing and retrieving other data;
c. a visible alphanumeric display means connected thereto,
d. a user input means adjacent to the display and connected to the processing means;
e. an alarm driven directly or indirectly from the CPU;
f. a pneumatic valve having an input port and an output port, and being driven directly or indirectly from the CPU; and
g. a program that runs on the CPU, for receiving user input, measuring input voltages and pressure voltages from the analog to digital converter, displaying data, converting measured voltages to gas concentration and pressure units, implementing a control algorithm, and driving the pneumatic valve and alarm.

2. The gas-based regulatory apparatus of claim 1, wherein the pneumatic valve input port is connectable to a user-supplied controlling gas source.

3. The gas-based regulatory apparatus of claim 2, wherein the pneumatic valve input port is connected to a pressure regulator which is in turn connected to the user-supplied controlling gas source.

4. The gas-based regulatory apparatus of claim 2, wherein the pneumatic valve input port is connected to a nitrogen source to regulate the oxygen content of a vessel in the hypoxic region.

5. The gas-based regulatory apparatus of claim 2, wherein the pneumatic valve input port is connected to an oxygen source to regulate the oxygen content of a control vessel in the hyperoxic region.

6. The gas-based regulatory apparatus of claim 1, further comprising an internal pressure means, thereby monitoring the constant availability of the gas.

7. The gas-based regulatory apparatus of claim 1 wherein the input port of the pneumatic valve is connected to the pressure measurement means.

8. The gas-based regulatory apparatus of claim 1 wherein the user input means are connected to the CPU such that numerical parameters, operating mode, and other relevant data can be entered into the memory of the CPU.

9. The gas-based regulatory apparatus of claim 1 wherein during normal operation the visual display shows the control setpoint and the present value of the controlled parameter.

10. The gas-based regulatory apparatus of claim 9, wherein the visual display additionally shows input gas line pressure and valve drive.

11. The gas-based regulatory apparatus of claim 1, wherein the visual display shows a menu to instruct the operator in the entry of at least one parameter.

12. The gas-based regulatory apparatus of claim 11, wherein the menu instructs the operator in the entry of a setpoint using the user input means.

13. The gas-based regulatory apparatus of claim 1, wherein the electrical input receives input from a sensor or analyzer that measures a variable that the user desires to control, with the analyzer or sensor either being within or sampling within a sealed or semi-sealed vessel within which the variable is to be controlled.

14. The gas-based regulatory apparatus of claim 1, wherein the electrical input is internally connected to a programmable gain amplifier, the gain of which is controlled via the CPU, and wherein an algorithm running in the program of claim 1 maximizes the resolution of the measured input voltage.

15. The gas-based regulatory apparatus of claim 14, wherein the algorithm accepts data between about 5 mV and about 5 V full scale, thereby enabling the use of raw oxygen fuel cell or other sensors, or conventional analyzers.

16. The gas-based regulatory apparatus of claim 1, wherein the measured value from a sensor or analyzer is optionally acquired in digital form via a digital data link.

17. The gas-based regulatory apparatus of claim 1, wherein the measured input is interactively transformed, via the user input means and the program, so that displayed units are familiar to the user.

18. The gas-based regulatory apparatus of claim 17, wherein the input varies from 0-5V and is converted to and displays as 0-100% O₂.

19. The gas-based regulatory apparatus of claim 17, wherein the transformed input is trimmed at the zero point and at a span point by interactive use of the user input means, program and display means.

20. The gas-based regulatory apparatus of claim 1, wherein a control algorithm running in the program reads the transformed or untransformed output of the attached analyzer or sensor, compares it to a user-entered setpoint, and implements a control algorithm of the Proportional Integral Derivative (PID) type.

21. The gas-based regulatory apparatus of claim 20, wherein the program controls the amount of the control gas per unit time passing through the pneumatic valve into a control vessel in accordance with the degree of delivery of the controlling gas dictated by the control algorithm.

22. The gas-based regulatory apparatus of claim 1, wherein the operator input accepts an alarm threshold for the variable being controlled, such that when the variable strays from the alarm threshold, the alarm is activated.

23. The gas-based regulatory apparatus of claim 22, wherein a plurality of alarm thresholds can be entered.

24. The gas-based regulatory apparatus of claim 1 further comprising a digital output means for retrieving data and transferring the data to an external memory.

25. The gas-based regulatory apparatus of claim 1 further comprising a digital input means or serial port for connecting the apparatus to an external computer, thereby enabling remote entry of data such as control setpoint and alarm thresholds.

26. The gas-based regulatory apparatus of claim 1 further comprising a drive for the pneumatic valve, the drive being connected to the CPU, thereby enabling a routine in the memory to detect an open circuit fault programmatically and optionally activate the alarm.

27. The gas-based regulatory apparatus of claim 1, wherein the zero and span of the pressure reading may be interactively adjusted by the user, using the user input and display means, such that the display reads 0 at 0 applied pressure to the control gas input port on the pneumatic valve, and the display reads the correct pressure when a known pressure is applied, with the sensitivity of the pressure measurement means being approximately 0-300 kPa.
